(19) United States
(12) Patent Application Publication
(10) Pub. No.: US 2019/0241852 A1
(43) Pub. Date: Aug. 8, 2019

(54) MICROFLUIDIC DEVICE WITH EXTRACELLULAR MATRIX SUPPORT MEMBRANE

(71) Applicants: Abhinav BHUSHAN, Chicago, IL (US); Chengyao WANG, Chicago, IL (US)

(72) Inventors: Abhinav BHUSHAN, Chicago, IL (US); Chengyao WANG, Chicago, IL (US)

(73) Assignee: ILLINOIS INSTITUTE OF TECHNOLOGY, CHICAGO, IL (US)

(21) Appl. No.: 16/270,376

(22) Filed: Feb. 7, 2019

Related U.S. Application Data
(60) Provisional application No. 62/627,375, filed on Feb. 7, 2018.

(51) Int. Cl. C12M 3/06; C12M 1/12

(52) U.S. Cl. CPC C12M 23/16; C12M 25/02

(57) ABSTRACT

A microfluidic device and method of use. A porous membrane is disposed between a top layer and a bottom layer, wherein the membrane separates, and is porous between, a first micrometer channel from a second micrometer channel. The first micrometer channel is etched in the top layer and the second micrometer channel is etched in the bottom layer each having a height of about 1 to 100 micrometers. The porous membrane can be a collagen membrane, and supports a cell culture.

### CROSS REFERENCE TO RELATED APPLICATION

This application claims the benefit of U.S. Provisional Application, Ser. No. 62/627,375, filed on 7 Feb. 2018. The co-pending Provisional Application is hereby incorporated by reference herein in its entirety and is made a part hereof, including but not limited to those portions which specifically appear hereinafter.

### FIELD OF THE INVENTION

This invention relates generally to microfluidic devices and, more particularly, to extracellular matrix membranous materials incorporated in microfluidic devices.

### BACKGROUND OF THE INVENTION

Knowledge derived from tissue engineering is increasingly applied in the development of micro-engineered models of human tissues and organs. These models, also called microfluidic devices, are being used as potential in vitro alternatives to animal models in simulating morphogenetic and pathogenetic processes, as well as drug screening platforms. Microfluidic devices are increasingly found in research centers, clinics and hospitals, contributing as more accurate and powerful tools for studying of drug delivery, monitoring of specific analytes, and medical diagnostics. The devices often introduce the third dimension (3D) to the in vitro cell cultures and are related to the integration of tissue engineering with microfabrication and microfluidics; advances in this field are associated with the convergence of biology with engineering.

Integration of cells cultured within a microengineered platform is often called an “organ-on-a-chip”. An organ-on-a-chip is a microfluidic device that contains continuously perfused chambers inhabited by living cells that allow modeling of the in vivo environment and enable precise control of cells and fluids to recapitulate the physiological and pathological conditions of complex tissues and organs. This technology is permissible to high-resolution, real-time imaging and in vitro analysis of biochemical, genetic and metabolic activities of living cells, which make them critical tools for finding functional properties, pathological states, and developmental studies of organs. There is thus a continuing interest and need for improved microfluidic devices.

### SUMMARY OF THE INVENTION

This invention includes an apparatus including a membrane made out of biological material, e.g., an extracellular matrix membranous material, in microfluidic devices, and methods of use and manufacture. The invention can be applied to incorporating such membranes in other devices, such as cell culture plates.

The invention includes a microfluidic device including a top layer, a bottom layer, and a porous membrane disposed between the top layer and the bottom layer. The membrane separates a first micrometer channel from a second micrometer channel. In embodiments of this invention, the first micrometer channel can have a height of 1 to 1000 micrometers, 1 to 500 micrometers, or 1 to 100 micrometers, between the top layer and the porous membrane, and the second micrometer channel can have a height of 1 to 1000 micrometers, 1 to 500 micrometers, or 1 to 100 micrometers, between the bottom layer and the porous membrane. Each micrometer channel is etched, molded, lithographically patterned, machined, laser-cut, or otherwise made in the corresponding layer.

The porous membrane supports a mammalian or bacterial cell culture, and can be a micrometer resolution membrane, desirably formed of a biological material. In embodiments of this invention, the porous membrane comprises a collagen membrane.

Collagen is a key element of basal lamina in physiological system that participates in cell and tissue culture. Its function is for cells maintenance, growth, angiogenesis, disease progression, and immunology. Embodiments of the present invention integrate a micrometer resolution membrane that is synthesized out of rat-tail type I collagen in a microfluidic device with apical and basolateral chambers. The collagen membrane can be generated by a lyophilization method.

In order to evaluate the compatibility of the resulted membrane with organs-on-chips technology, it was sandwiched between the layers of polydimethylsiloxane (PDMS) that had been prepared by replica molding and the device was used to culture human colon caco 2 cells on the top of the membrane. Membrane microstructure, transport, cells viability and immunofluorescence microscopy (tight junction (Zonula Occludens-1), ezrin (Villin 2) and F-actin) in the organs-on-chips were observed to confirm the suitability of our resulted membrane. Through transport studies, the diffusion of two different membranes, Transwell and the collagen membrane of this invention, were compared. The mass transport of 40 kDa dextran was found to be an order of magnitude higher through the collagen membrane than that through the Transwell membrane. Human colon caco 2 cells were cultured in devices with no-, Transwell, and ECM membrane to evaluate the compatibility and proteins expression of cells on ECM membrane compared to other two membranes. Live/dead and immunofluorescence analysis results revealed that the caco 2 cells cultured on the collagen membrane had excellent viability and function for extended periods of time and displayed stronger fluorescence signals in tight junction (ZO-1), ezrin (Villin 2), F-actin compared to other two devices. The results indicate a substantial improvement in establishing a physiological microenvironment for in vitro organs-on-chips.

### BRIEF DESCRIPTION OF THE DRAWINGS

FIG. 1 is an exploded perspective view of a microfluidic device according to one embodiment of this invention.

FIG. 2 is a sectional side view of a of a microfluidic device according to one embodiment of this invention.

FIG. 3 shows a collagen membrane fiber structure by confocal microscope (scale bar, 100 um), with a magnified view of clustered fibers and pores of the collagen membrane (scale bar, 20 um).

FIG. 4 shows a microfluidic device with no membrane (top), a sandwiched Transwell membrane (middle) and a sandwiched collagen membrane according to this invention (bottom).

FIG. 5A shows a microfluidic device with an extracellular matrix membranous material according to this invention.

FIG. 5B shows a microfluidic device with a Transwell membrane.

FIG. 5C shows a microfluidic device with no membrane.

FIG. 6 summarizes dextran concentration in a bottom chamber of devices as a function of time (n=3), where circles indicate a device with the collagen membrane, and rectangles indicate a device with a Transwell membrane.

FIG. 7 shows live and dead stains (calcein-AM and ethidium homodimer-1) of caco 2 cells over a course of 5 days seeded in the devices with no membrane, a Transwell membrane, and a collagen membrane, respectively (Scale bars, 100 um).

FIG. 8 is a graph reporting viability of caco 2 cells in the tested microfluidic devices (no membrane, Transwell membrane, and collagen membrane) for 5 days (n>6).

FIG. 9 shows immunofluorescent images of caco 2 cells in devices with no, Transwell, and collagen membranes, respectively. F-actin, tight junction and ezrin in caco 2 cells were stained by Phalloidin, ZO-1, and Ezrin (Scale bar, 20 um). Three different cell morphologies were marked as: squamous—white arrows; round—dark arrows; and cells that appeared integrated with collagen fibers—light grey arrows. Quantification of the expression of proteins for the three devices (** p<0.001) is also shown.

### DETAILED DESCRIPTION OF THE INVENTION

The present invention provides a device incorporating a membrane made out of biological material. Embodiments of this invention include a collagen membrane incorporated in a microfluidic device that is useful for supporting mammalian cell culture and research in vitro. The two-chamber microfluidic device includes two polymer layers—top layer and bottom layer, and the layers sandwich a collagen membrane which separates the channel to two chambers. Collagen is one type of useful extracellular matrix (ECM) protein, and can be prepared through a modified freeze drying process.

The device of embodiments of this invention supports cell culture. The microfluidic device and methods of this invention can be used in, for example, cell and tissue culture including organs-on-chips that are used to study metabolism, bacterial interactions, and drug development.

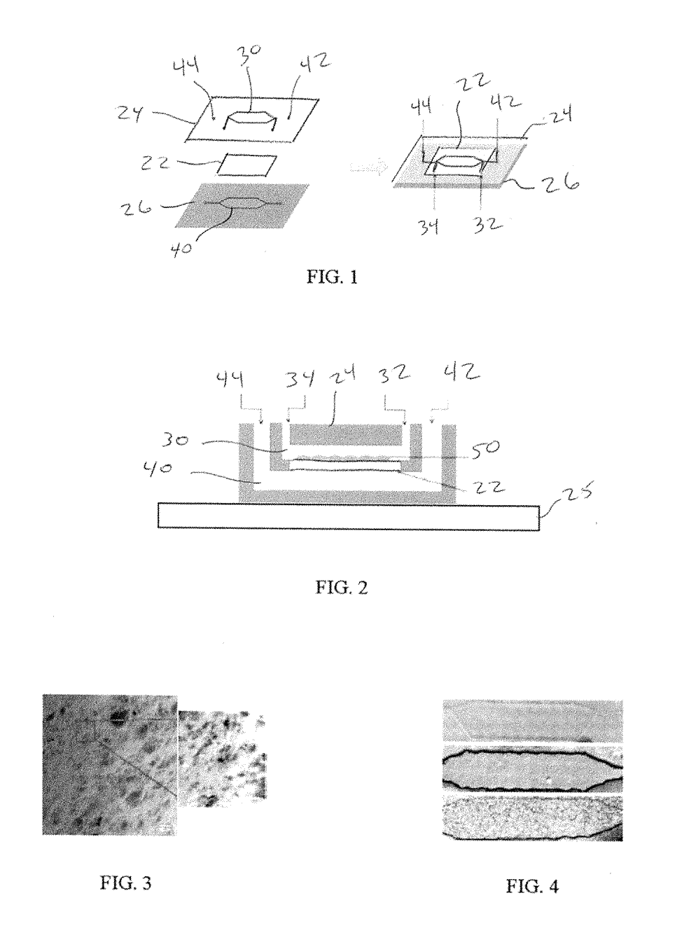

FIGS. 1 and 2 show the microstructure of a microfluidic device 20 according to embodiments of this invention. A collagen membrane 22 is sandwiched between a top layer 24 and a bottom layer 24. The structure can be built on, for example, a glass slide 25. The membrane 22 separates a first micrometer channel 30 from a second micrometer channel 40. As shown in FIG. 1, the first micrometer channel 30 is desirably etched in the top layer 24 and the second micrometer channel 40 is desirably etched in the bottom layer 26. The first micrometer channel 30 includes an inlet 32 and an outlet 34. The second micrometer channel 40 also includes an inlet 42 and an outlet 44. The porous membrane supports a cell culture 50.

FIG. 3 shows a collagen membrane fiber structure of this invention by confocal microscope (scale bar, 100 um), with an exploded view of clustered fibers and pores of the collagen membrane (scale bar, 20 um). FIG. 4 shows a microfluidic device with no membrane (top), a sandwiched Transwell membrane (middle) and a sandwiched collagen membrane according to this invention (bottom).

In embodiments of this invention, the microfluidic chips are fabricated out of polyester, polycarbonate, and/or preferably polydimethylsiloxane (PDMS), owing to the material's chemical inertness, and biocompatibility. The devices can contain multiple layers such as the top layer, the bottom layer, and the porous membrane that is sandwiched between the two layers. Due to the oxygen permeability of PDMS, the cells can be either cultured on the bottom layer or on the membrane in the microfluidic devices.

Embodiments of this invention include microfluidic devices that incorporate a micrometer resolution membrane that is synthesized out of rat tail type I collagen. Type I collagen was coated on a glass slide and lyophilized overnight. The membrane was then peeled off, cut to appropriate size and sandwiched between the layers of PDMS that had been prepared by replica molding. The resulting device had two independent channels separated by the collagen membrane, such as shown in FIGS. 1 and 2.

The present invention is described in further detail in connection with the following examples which illustrate or simulate various aspects involved in the practice of the invention. It is to be understood that all changes that come within the spirit of the invention are desired to be protected and thus the invention is not to be construed as limited by these examples.

### EXAMPLES

This example includes the development and characterization of a multilayer microfluidic device that incorporates a novel membrane, which is made out of, type I collagen. Type I collagen is one of the most abundant proteins in the extracellular matrices in vivo and is widely used as a supporting matrix. Type I collagen is the primary constituent of the major ECM proteins in the intestine involved in several cellular processes including remodeling, wound healing, metastasis, the microenvironment. Type I collagen coatings on well-plates are used to support adhesion, and function of intestinal cells including caco 2 and crypts. Recently developed lyophilized collagen layers and scaffolds have shown superior cellular response and function over PDMS layers, however, these collagen structures have not been incorporated in any microfluidic organotypic device. The invention includes a process to incorporate the membrane into two-chamber microfluidic devices, wherein the devices are biocompatible, and the example compares the cell viability with conventional microfluidic devices. Caco 2 cells were used, as they have been used to model the human colon in both standard tissue culture plates, and microfluidic devices.

### Methods and Materials

### Collagen Membrane

10×DMEM (Gibco) was adjusted to pH 7.3 by adding saturated sodium bicarbonate (Sigma). Rat-tail type I collagen solution (BD Biosciences, 3.30 mg/ml) was mixed with the 10×DMEM at ratio of 9:1, and pH adjusted to 7.2. The collagen solution was spin coated on a glass slide, frozen at 4, −20, −80° C. for 6 hours separately and placed in a lyophilizer (LABCONCO, Kansas City, Mo.) overnight. The lyophilized collagen membrane was peeled off and cut to the required shape needed for the microfluidic devices. The fibrous microstructure of the collagen membrane was characterized under a confocal microscope (Carl Zeiss Microscopy, Thornwood, N.Y.) (FIG. 3).

### Fabrication of the Microfluidic Device

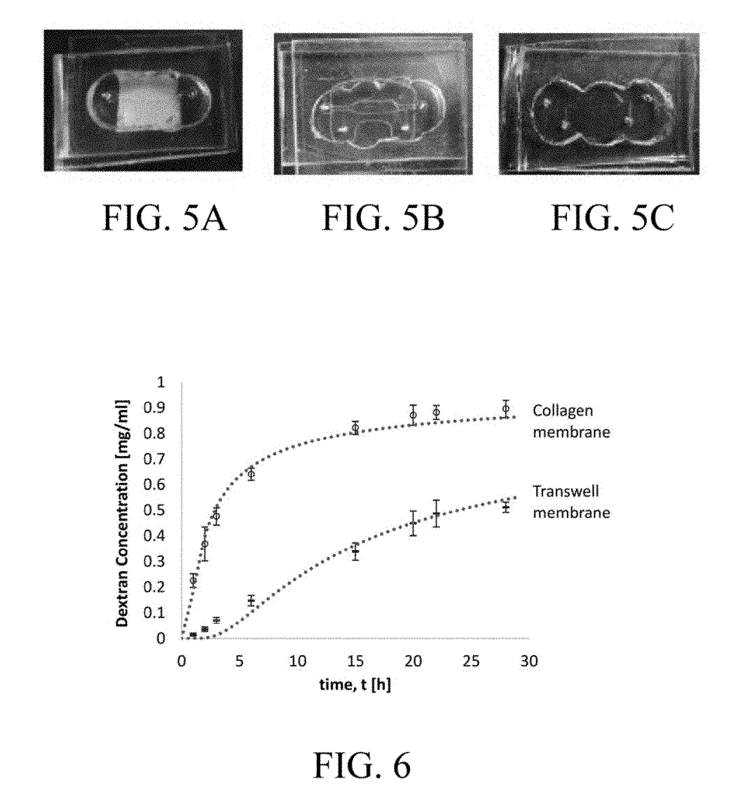

Three types of microfluidic devices were fabricated (FIGS. 4 and 5), each with different membranes: (1) a single-chamber device with no membrane, (2) a two-chamber device with Transwell polyester membrane (10 um thickness, 0.4 um pore size, Corning, N.Y.), and (3) a two-chamber device with the collagen membrane. The single chamber device was fabricated using standard soft lithography protocols. The two-layer device with the Transwell membrane was fabricated using a method developed before. To fabricate the device with the collagen membrane, PDMS monomers (Dow Chemical Co, Midland, Mich.) were mixed and put in replica mold, vacuumed to remove bubbles inside the PDMS, and put in an oven at 70° C. The bottom layer was made out of a 250 um thick PDMS sheet that was cut by a laser cutter. Both the PDMS layers were treated in plasma cleaner (Harrick Plasma, Ithaca, N.Y.). The bottom PDMS layer was first bound to a cleaned microscope glass slide. The lyophilized collagen membrane temporarily adhered to the plasma-cleaned top PDMS layer electrostatically. The plasma-cleaned top PDMS layer with the collagen membrane side was inverted, placed on the bottom layer, and then bonded together using the plasma cleaner so that the collagen membrane was securely sandwiched between the two PDMS layers (FIG. 1).

Mass Transport Characteristics of the Membrane in the Device

Mass transport through the collagen membrane was compared to the Transwell membrane by studying diffusion of FITC dextran (40 kDa molecular weight, Sigma Aldrich, USA) in the two devices. The fluorescence of the transported molecule was measured by a microplate reader (SpectraMax M2, Molecular Devices, Sunnyvale, Calif.). Devices were assembled, following the protocols described above, each one integrating the Transwell membrane or the collagen membrane. FITC dextran solution (2 mg/mL in DI water) was filled in the upper chamber of the devices, while the lower channels were only filled with DI water. Devices were protected from light throughout the experiment. The liquid from the lower channels was collected after 1, 2, 3, 6, 15, 20, 22 and 28 hours. The collected samples were transferred to a 96-well plate and the fluorescence was read by the microplate reader. Three devices were used for each time point. A standard curve relating the fluorescence of FITC-dextran versus concentration was also constructed. As previous work shows, the linear standard curve of dextran concentration and fluorescence is valid at a low dextran concentration. Therefore, the concentration of FITC-dextran was ranged to 0.01, 0.001 and 0.0001 mg/ml and plotted the standard curve. The concentration of samples was read from the standard curve.

The device was modeled as a closed system; in other words, the top and the bottom chambers were taken together as the system since there were no losses out of the device during the experiment. In this case, the total concentration of FITC-dextran in the system was C₀, or 2 mg/ml. Cₜ (t) was the concentration in the top chamber and C_{b} (t) was the concentration in the bottom chamber. Then,

C₀=Cₜ(t)+C_{b}(t)

The boundary conditions and initial conditions correspond to

C(x≥0,t=0)=0, (2)

C(X→∞,t≥0)=0, (3)

and C(x=0,t≥0)=C₀−C_{b}(t). (4)

To obtain the diffusion coefficient of dextran through membranes, the one-dimensional (1D) Fick's law of diffusion was used (Eq. 5).

$$\frac{\_C ( x , t )}{\_t} = D \frac{{\_}^{2} C ( x , t )}{{\_x}^{2}} \qquad ( 5 )$$

where C (x, t) is the concentration of diffusing particles (dextran) as a function of time (t) and depth (x), and D is the diffusion coefficient of dextran though the membranes.

Eq. 5 was solved with these conditions and the solution obtained as Eq. 6.

$$C ( x , t ) = {C}_{0} \frac{erfc \frac{x}{2 \sqrt{Dt}}}{1 + erfc \frac{x}{2 \sqrt{Dt}}} \qquad ( 6 )$$

where er f c is the error function complement. The depth x in Eq. 2 was adapted to the thickness l of membrane which modified Equation 6 to

$${C}_{l} ( t ) = {C}_{0} \frac{erfc \frac{l}{2 \sqrt{Dt}}}{1 + erfc \frac{l}{2 \sqrt{Dt}}} \qquad ( 7 )$$

where l is the thickness of the membrane and Cₗ(t) is the dextran concentration at x=l, which is approximately 15 um for collagen membrane and 10 um for Transwell membrane. The model is appropriate due to interest in determining the transport across the membrane, in other words, at a fixed x=l. Eq. 7 was fitted to the average concentration data as a nonlinear least-square fit by Matlab (The Mathworks, Natick, Mass.). The curve fitted plot is shown in FIG. 6.

### Cell Culture in Microfluidic Device

Caco 2 cells were seeded in the three devices. Before seeding cells, devices were sterilized under UV light for 15 min. The glass surface of the one-chamber device and the membrane of two-chamber devices were coated by 50 ug/ml fibronectin for 30 min at 37° C. in incubator. Then, 100 ug/ml type I collagen solution was used to coat the single-chamber device and the two-chamber device with polyester membrane for 30 min at 37° C. in incubator. Caco 2 cells were then seeded in the devices. The cells were cultured for up to days and the media was changed daily. The cells viability was assessed by using a live/dead staining kit (calcein-AM and ethidium homodimer-1, Thermo Fisher); the live and dead cells were quantified using ImageJ (NIH, Bethesda, Md.).

### Immunofluorescence Microscopy

For immunofluorescence microscopic analysis, cells grown in the microfluidic devices were fixed with 4% (w/v) PFA (paraformaldehyde). For the F-actin and ezrin stains, fixed cells were permeabilized with 0.5% (v/v) Triton X-100; for tight junction staining, the fixed cells were not permeabilized as the protein is located at membrane. Then cells were blocked with 3% (w/v) bovine serum albumin (BSA) and washed with phosphate buffered saline (PBS, Ca′ and Ma′ free; Gibco). The fixed specimens were then incubated with the conjugated antibody dissolved in 1% BSA at 4° C. overnight, under light protected conditions. The antibodies for each target protein were obtained as follows: Phalloidin (Biotium, Hayward, Calif.), anti-ZO-1 (rabbit polyclonal; Bioss, Woburn, Mass.), and ezrin (rabbit polyclonal; Novus Biologicals, Littleton, Colo.). At least three replicate microfluidic devices were analyzed in each experiment and recorded images at more than three randomly selected sites, and each experiment was repeated at least twice. The images were taken using a Zeiss laser scanning confocal microscope and the fluorescent intensity was quantified using ImageJ.

### Statistical Analysis

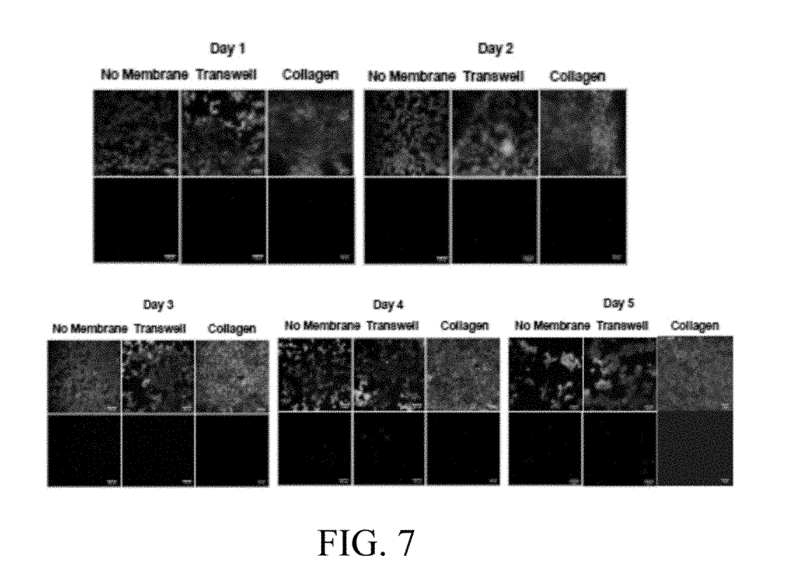

All results in this paper are reported as mean±standard error. For statistical analyses in FIG. 7, a one-way analysis of variance (ANOVA) with Tukey-Kramer multiple comparisons test was used. Differences between groups were considered statistically significant when p<0.001.

### Results—Type I Collagen Membrane

The fibers of the type I collagen fibers on the membrane in the microfluidic devices were well organized and formed a dense reticular structure (FIG. 3). As the detailed clustered fibers and pores of membrane show, the membrane is evenly woven (FIG. 3). The clustered and densely knitted collagen fibers provided a higher contact area for cells than membranes coated with collagen. The diameter of collagen fibers in the membrane was estimated at ˜1.2 um, the diameter of the fiber clusters varied from 1 to 20 um.

### Mass Transport Characteristics of the Membrane in the Device

The mass transport and the model are shown in FIG. 2. By using Matlab to fit the experimental data to developed Eq. 7, the diffusion coefficient of dextran was obtained through the collagen membrane as 4.191×10⁻⁷ cm²/s and that through the Transwell membrane as 2.242×10⁻⁸ cm²/s. The average pore diameter of the collagen membrane was estimated to be 10.2 um. The larger pore size of the collagen membrane than the Transwell membrane could explain the higher mass transport. The experimental data can now be used to determine transport of proteins and other molecules through the collagen membrane.

### Live/Dead Analysis

The live/dead staining results show the growth and death of caco 2 cells in the three devices (FIG. 7). For the single chamber device with no membrane, caco 2 cells were confluent by day 3; however, after that, the viability declined and on day 5, the number of cells was lower than the initial seed. For the device with polymer membrane, caco 2 cells multiplied until day 3, however, the number of live cells dropped beyond day 3; the rate of decline in the number of live cells was lower in this case than that in the single chamber device. In contrast, the cells in device with collagen membrane showed a steady increase in the number of live cells with no decline even on day 5. The change in the number of dead cells in the devices differed from the growth of cells. For the device without a membrane, there were fewer dead cells until day 2; however, beyond that, there was an increase in the number of dead cells. The device with the Transwell membrane showed a similar pattern. The morphology of caco 2 cells growing on glass and the membranes was quite different between the three devices. Most of caco 2 cells seeded on glass in the single chamber device with no membrane kept the original rounded shape, and even when they are confluent, the adjacent cells do not change their shape. Cells seeded on the Transwell membrane initially had a rounded morphology, however, when the cells started to become confluent, the morphology became elliptical.

On the collagen membrane device, there are three different morphologies—round, squamous and collagen fibers integrated. In the first couple of days, the cells that were not in contact with another cell maintained a round morphology whereas those in contact with the other cells took a squamous appearance. As the cells became confluent on subsequent days, more cells acquired a squamous appearance and the cells along the collagen fibers appeared to integrate with the collagen fibers. In fact, on day 5, the cells grew into more epithelium and appeared to remodel the microenvironment. Live/dead staining results (FIG. 7) indicated that while the cells on the glass and the polymer membrane start to become apoptotic, the cells on the collagen membrane remained viable.

### Cell Viability

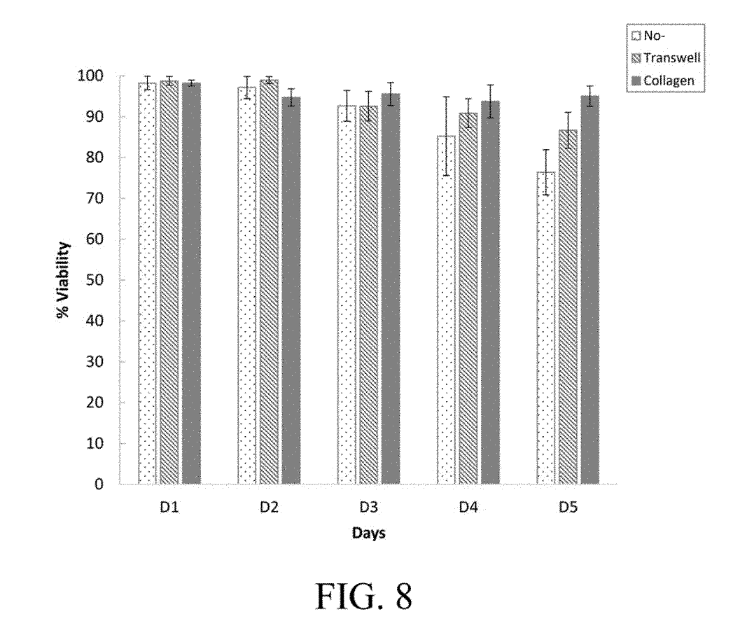

The viability of cells seeded in the three types of devices is shown in FIG. 8. Viability of cells in the device with no membrane decreased steadily after day 1, with the day 5 viability being 76%. For the device with the Transwell membrane, although the cells started with a high viability, the viability decreases steadily subsequently with a day 5 viability of 85%. The viability of the caco 2 cells on the collagen membrane declined initially but increased to >95% on day 5. Overall, the collagen membrane does not affect the cell viability, in fact, it appears that the cells viability is the most stable when on the collagen membrane.

### Immunofluorescence Microscopy

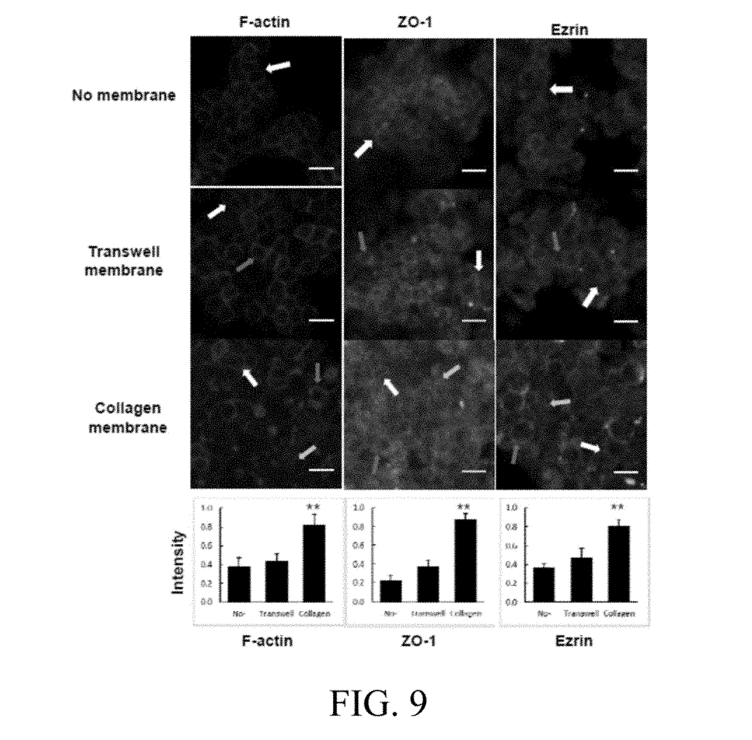

Immunofluorescence microscopy revealed that the caco 2 cells in the devices with collagen membrane had higher expression of F-actin, ezrin, and tight junction (ZO-1) compared to devices with no membrane and Transwell membrane (FIG. 9). The normalized fluorescence intensity for each of the three proteins was higher in the device with collagen membrane than the other two devices. The cells in device with no membrane were mostly squamous shaped (marked with white arrows), those in the device with Transwell membrane were squamous and rounded (marked with dark arrows), whereas those in the device with the collagen membrane were shaped squamous, rounded, as well as cells that appeared integrated with the collagen fibers (light grey arrows).

### Example Discussion

In vitro models of the basal membranes, such as polyester and Transwell, are common in cell biology and organs-on-chips devices where cells require apical and basolateral polarization. Organs-on-chips seek to understand complex processes such as organ development, cell-cell interactions, embryogenesis, and diseases that are regulated by cellular responses to multiple chemokines. While the selection of biocompatible polymer membranes is wide in microfluidic device, the lyophilized ECM membranes represent a technological challenge and are not applied in organs-on-chips. Well-patterned fabrication of ECM membranes and embedded in microfluidic device are difficult by current method. First of all, due to surface tension and viscosity of the ECM solution, it is difficult to spread and fill the surface of the mold completely and homogeneously. Furthermore, mechanical peeling of the membrane is problematic as the thickness and fragility of the lyophilized membrane. Once peeled from the glass slide, the membrane must be cut precisely, transferred and attached to the microfluidic device without any wrinkles and folds. The invention provides a novel integration of ECM membrane into a microfluidic device. Rat type I collagen was selected in the example since its formation and function for tissue maintenance, growth, angiogenesis, disease progression, and immunology are well studied.

The method of this example provides a quick, repeatable, and convenient route to prepare desired ECM membranes. First, the method can make dense ECM fibers and well-kilted ECM membrane with uncomplicated process that makes this method can be widely applied in industrial production of ECM membrane. The observation under microscope shows that the lyophilized type I collagen fibers in microfluidic device were well organized and evenly woven so that formed a dense reticular structure. The average pore diameter of the collagen membrane appears larger than the Transwell membrane; and so that the mass transport of collagen membrane in device is higher than with the Transwell membrane. The ECM membrane embedded in microfluidic device separates the device into two fluidic chambers and particulates in chambers are able to pass through the porous membrane. This is an essential characteristic in organs-on-chips technology that cellular extravasation and passage of chemical species can interact between communicating chambers.

Further, while the biocompatible polymer membranes are widely used in organs-on-chips, they cannot be embedded in vivo. The purpose of organs-on-chips technology is establishing an in vitro method to understand complex processes such as organ development, cell-cell interactions, embryogenesis, study and finally find ways to cure diseases that are regulated by cellular responses. The result shows that the ECM membrane is more biological than Transwell and polyester membrane. To evaluate the compatibility of cells on ECM membrane compared to other two membranes, human colon caco 2 cells were cultured in devices with no, Transwell, and ECM membrane. The viability and morphology of caco 2 cells were then observed. The live/dead analysis and cells viability results show the growth and apoptosis of caco 2 cells cultured on the membrane. After caco 2 cells were confluent, while the cells on the glass and the polymer membrane start to become apoptotic, the cells on the collagen membrane remained viable. The cells viability result sustains this observed result; it shows that while cells viability of cells in device with no membrane reduced to 76% and Transwell membrane decreased to 85% at day 5, device with collagen membrane remained at around 95%. By comparing cell cultured in the device with no membrane and Transwell membrane, different cell morphology on the no-, Transwell, and collagen membrane were observed. Unlike the other two devices that only round and squamous morphology were observed, collagen fibers integrated morphology was also observed in device with collagen membrane. When caco 2 cells were confluent in device with collagen membrane, the cells along the collagen fibers appeared to integrate with the collagen fibers and then the cells grew into more epithelium and appeared to remodel the microenvironment. This data indicates that the device with collagen membrane supports growth and viability of the cells over the other two devices. The differences in the cell morphologies that was observed were supported by the immunofluorescence images—cells on the collagen membrane displayed more tight junction, ezrin and F-actin proteins. On the collagen membrane, cells not only showed round and squamous shapes but also appeared to integrate with the fibers. The data shows that the cells on the collagen membrane expressed the three proteins (F-actin, ZO-1, and ezrin) several folds higher than the other two devices. Since these proteins play important roles in cell function and tissue engineering (e.g., actin participates cell division and maintenance of cell junctions among other functions, tight junction proteins regulate the barrier function, and ezrin plays a key role in cell surface structure adhesion, migration, and organization), the data suggests that the collagen membrane provides the cells with a much improved microenvironment.

Advantages of embodiments of this invention include: it uses a simple fabrication process that can be easily integrated with multilayer microfluidic devices, and the devices are closer physiologically in mimicking the microenvironment than those with a polymer (Transwell) membrane. The membrane was characterized and the mass transport of high molecular weight fluorescent molecule was quantified, which can now be used to predict the transport of proteins through the membrane. It was shown that cells cultured on the collagen membrane do not lose viability compared to those in the devices with no- or Transwell membranes. In addition, integration of the cells with the collagen fibers was an interesting observation, which indicated that the collagen membrane provided a more physiological microenvironment to the cells.

Thus, the invention provides a microfluidic device with a collagen membrane that can be used to study cell-cell interactions and drug metabolism. Because of the wide use of type I collagen in cell culture and the growing applications of microfluidic based microphysiological systems, the device with the collagen membrane will be widely applicable to many research areas.

The invention illustratively disclosed herein suitably may be practiced in the absence of any element, part, step, component, or ingredient which is not specifically disclosed herein.

While in the foregoing detailed description this invention has been described in relation to certain preferred embodiments thereof, and many details have been set forth for purposes of illustration, it will be apparent to those skilled in the art that the invention is susceptible to additional embodiments and that certain of the details described herein can be varied considerably without departing from the basic principles of the invention.

What is claimed is:

1. A microfluidic device, comprising:
a top layer and a bottom layer;
a porous membrane disposed between the top layer and the bottom layer, wherein the membrane separates a first micrometer channel from a second micrometer channel.

2. The device of claim 1, wherein the first micrometer channel has a height of 1 to 1000 micrometers between the top layer and the porous membrane, and the second micrometer channel has a height of 1 to 1000 micrometers between the bottom layer and the porous membrane.

3. The device of claim 1, wherein the first micrometer channel is etched, molded, lithographically patterned, machined, laser-cut, or otherwise made in the top layer and the second micrometer channel is etched, molded, lithographically patterned, machined, laser-cut, or otherwise made in the bottom layer.

4. The device of claim 1, wherein the porous membrane supports a mammalian or bacterial cell culture.

5. The device of claim 1, wherein the porous membrane comprises a collagen membrane.

6. The device of claim 1, wherein the porous membrane comprises a micrometer resolution membrane.

7. The device of claim 1, wherein the porous membrane comprises a biological material.

8. The device of claim 1, wherein each of the top layer and the bottom layer comprises polydimethylsiloxane.
